(19) United States
(12) Patent Application Publication
(10) Pub. No.: US 2005/0009026 A1
(43) Pub. Date: Jan. 13, 2005

(54) Surface for the immobilisation of nucleic acids

(76) Inventors: Andreas Peter Abel, Basel (CH); Markus Ehrat, Magden (CH); Ekkehard Kauffmann, Basel (CH); Dominic Utinger, Etingen (CH); Vincent Benoit, Saint louis (FR); Susan Margaret De Paul, Zurich (CH); Marcus Textor, Schaffhausen (CH); Stephanie Vande Vondele, Cambridge (GB); Jeffrey Alan Hubbell, Morges (CH)

Correspondence Address: WENDEROTH, LIND & PONACK, L.L.P., 2033 K STREET N. W., SUITE 800, WASHINGTON, DC, 20006-1021, US

(21) Appl. No.: 10/487,915

(22) PCT Filed: Aug. 24, 2002
(86) PCT No.: PCT/EP02/09490

(30) Foreign Application Priority Data
Aug. 27, 2001 (CH) 1586/01

(51) Int. Cl. A61L 2/00; C12Q 1/68; C07H 21/04; C12M 1/34

(52) U.S. Cl. 435/6; 435/287.2; 536/25.3; 427/2.11

(57) ABSTRACT

The invention relates to a surface for the immobilization of one or several first nucleic acids as recognition elements (“immobilization surface”), for the production of a recognition surface for the detection of one or several second nucleic acids in one or more samples which are brought into contact with the recognition surface, the first nucleic acids being applied to a layer of the graft copolymer poly(L-lysine)-g-poly(ethyleneglycol) (PLL-g-PEG) as surface for immobilization, characterized in that the grafting ratio g, in other words the ratio between the number of lysine units and the number of polyethylene glycol side chains (“PEG” side chains) has an average value between 7 and 13. The invention also relates to a method for the qualitative and/or quantitative detection of one or more second nucleic acids in one or more samples, characterized in that said samples and optionally further reagents are brought into contact with an immobilization surface according to the invention, upon which one or several first nucleic acids are immobilized as recognition elements for specific binding/hybridization with said second nucleic acids and changes in optical or electronic signals resulting from the binding/hybridization of said second nucleic acid, or further, resulting from applied tracer substances applied for analyte detection, are measured.

The invention in hand relates to a surface for the immobilization of one or several first nucleic acids as recognition elements (“immobilization surface”), for the production of a recognition surface for the detection of one or several second nucleic acids in one or more samples which are brought into contact with the recognition surface, the first nucleic acids being applied to a layer of the graft copolymer poly(L-lysine)-g-poly(ethyleneglycol) (PLL-g-PEG) as surface for immobilization, characterized in that the grafting ratio g, in other words the ratio between the number of lysine units and the number of polyethylene glycol side chains (“PEG” side chains) has an average value between 7 and 13. In the following, the “immobilization surface” as defined above, together with the first nucleic acids immobilized thereon, is called a “recognition surface”.

The invention also relates to a method for the qualitative and/or quantitative detection of one or more second nucleic acids in one or more samples, characterized in that said samples and optionally further reagents are brought into contact with an immobilization surface according to the invention, upon which one or several first nucleic acids are immobilized as recognition elements for specific binding/hybridization with said second nucleic acids and changes in optical or electronic signals resulting from the binding/hybridization of said second nucleic acid, or further, resulting from applied tracer substances applied for analyte detection, are measured.

In the context of the invention in hand, the term “nucleic acids” shall mean single- or double-stranded compounds from the group formed by oligonucleotides, polynucleotides, DNA or RNA strands and DNA or RNA analogs, e.g. comprising modified bases or “backbones”. In this definition of the term “nucleic acids” shall also be included hybrids of DNA and RNA and their analogs.

For the detection of one or more analytes from a sample with a complex mixture of numerous substances there are widespread methods in which one or more so-called recognition elements which are of biological, biochemical or synthetic character are immobilized on a solid carrier before they are then brought into contact in immobilized form with said sample and the analytes contained therein bind to the recognition elements specific for them. In this case, the solid carrier may be both of macroscopic nature with a surface of square millimeters to square centimeters or also of microscopic nature, for example in the form of so-called beads, i.e. approximately spherical particles with typical diameters in the micrometer range. The surface of such a solid carrier with recognition elements immobilized thereon shall hereinafter be called a “recognition surface”.

Compared with methods in which the analytes and their recognition elements are brought together as reaction or binding partners in homogeneous liquid solution, these methods which are based on a solid carrier offer numerous advantages, for example an easier separation or differentiation of bound analyte molecules from the sample matrix. These advantages are gained with a restriction of the diffusion-driven mixture between analyte molecules and recognition elements, because of the binding of the recognition elements to the solid carrier.

For the preparation of recognition surfaces for the highly efficient and highly selective binding of the one or more analytes to be detected in a sample, the quality of these surfaces is of major importance. To achieve the lowest possible limits of detection, it is desirable to immobilize in a small space as many recognition elements as possible in such a way that as many analyte molecules of one variety as possible may then be bound in the later detection process. At the same time it is desirable on immobilization to maintain as high a degree of reactivity and biological or biochemical functionality of the recognition elements as possible, i.e. to minimize any signs of denaturation resulting from the immobilization. A further objective is as far as possible to prevent the nonspecific binding or adsorption of analyte molecules which in many cases have the effect of restricting the limits of detection attainable.

Especially for the analysis of nucleic acids, microarrays with a partially very high “feature density”, i.e. density of discrete measurement areas comprising biological or biochemical recognition elements immobilized therein on a common carrier, are known since about 1990.

Within the terms of the present invention, laterally separate measurement areas, as an integral part of a recognition surface, shall be defined by the surface area which encompasses the biological or biochemical or synthetic recognition elements immobilized thereon for the detection of an analyte from a liquid. These areas may be present in any geometric form, for example in the form of points, circles, rectangles, triangles, ellipses or lines. It is possible that up to 1,000,000 measurement areas may be present in a two-dimensional arrangement, wherein a single measurement area may take up an area of 0.001 mm²-6 mm². The density of the measurement areas may typically amount to more than 10, preferably more than 100, especially preferably more than 1000 measurement areas per square centimeter.

In the following, an array shall mean a two-dimensional arrangement of measurement areas on a common carrier. Thereby, the carrier may have an essentially planar or also any other, for example spherical geometry.

In U.S. Pat. No. 5,445,934 (Affymax Technolgies), for example, arrays of oligonucleotides arranged at a density of more than 1000 features per square centimeter are described and claimed.

For an improvement of the adhesion and stability of the immobilization of biological, biochemical or synthetic recognition elements it is often advantageous to deposit initially a so-called adhesion-promoting layer on the carrier. The adhesion-promoting layer may comprise, for example, chemical compounds compound from the group of silanes, functionalized silanes, epoxides, functionalized, charged or polar polymers and “self-assembled passive or functionalized monolayers or multilayers”. Such adhesion-promoting layers and specific requirements on the properties of an adhesion-promoting layer, which are dependent on the physical and chemical type of the carrier and the related measurement arrangement, are described, for example, in the patent applications WO 95/33197, WO 95/33198, WO 96/35940, WO 98/09156, WO 99/40415, PCT/EP 00/04869, and PCT/EP 01/00605.

In U.S. Pat. Nos. 5,820,822, 5,232,984, 5,380,556, 6,231,892, 5,462,990, 5,627,223, and 5,849,839 graft copolymers are described which comprise a charged, poly-ionic main chain and bound thereto (“grafted,”) “non-interactive” (adsorption-resistant, uncharged) side chains. For example, the production of so-called “bio-compatible” surfaces of so-called “micro-capsules” to be applied in vivo or of implants is described as application of such polymers. Thereby, the term “bio-compatibility” is applied in the meaning of the ability of preventing or, at least, minimizing the adhesion of cells or proteins to such coated surfaces, which could, e.g., lead to an immune defense or to a final rejection of an implant in a living organism. This property is achieved upon promoting by electrostatic interaction the adhesion of the charged polymer main chain to an oppositely charged surface of the carrier to be coated, and enabling the adhesion of biomolecules by means of the “non-interactive” (uncharged) side chains.

Applications of such polymer coatings in bio-analytics, e.g. for the production of an adhesion-promoting layer for the immobilization of biological recognition elements on a sensor platform, are described in WO 00/65352. Here poly(L-lysine)-g-poly(ethyleneglycol) (PLL-g-PEG) is preferred as a graft co-polymer. In this context, g annotates the grafting ratio, i.e. the ratio between the number of lysine units and the number of polyethylene glycol side chains (“PEG” side chains).

As mentioned in WO 00/65352, the optimum value of g is always dependent on the size of the PEG side chains and the application under consideration. Optimum values of 3<g<10, preferably of 4<g<7, for PEG chains with a molecular weight of 5000 Da, and of 2<g<8, preferably of 3<g<5, for a PEG molecular weight of 2000 Da, are specified in WO 00/65352. These values in WO 00/65352 are related to the minimization of nonspecific binding to a surface coated with PLL-g-PEG, the surface being dedicated for the detection of proteins by means of sensors whereon the analyte-specific recognition elements had been immobilized on a PLL-g-PEG coated surface.

Surprisingly, it has now been found that optimum ratios between specific and non-specific binding (or specific and non-specific hybridization, respectively), for the detection of nucleic acids in nucleic acid hybridization assays using nucleic acids, immobilized as recognition elements (here also called “capture probes”) on a surface coated with PLL-g-PEG, are achieved at average values of g between 7 and 13.

Therefore, a first subject of the invention is a surface for the immobilization of one or several first nucleic acids as recognition elements for the production of a recognition surface for the detection of one or several second nucleic acids in one or more samples which are brought into contact with the recognition surface, the first nucleic acids being applied to a layer of PLL-g-PEG as a surface for immobilization, characterized in that the grafting ratio g has an average value between 7 and 13.

Thereby it is preferred that the grafting ratio g has a medium value between 8 and 12.

It is preferred simultaneously that the molecular weight of the polyetheyleneglycol side chains (“PEG” side chains) is between 500 Da and 7000 Da. Especially preferred is if the molecular weight of the PEG side chains is between 1500 Da and 5000 Da.

Preferably, the surface for the immobilization of one or several first nucleic acids, according to the invention, is deposited on a solid carrier. This carrier is preferably essentially optically transparent.

The term “essentially optically transparent” is understood to mean that carriers or layers thus characterized are a minimum of 95% transparent at least at the wavelength of light delivered from an external light source for its optical path perpendicular to said carrier or layer, respectively, provided the carrier or layer is not reflecting. In the case of partially reflecting carriers or layers, “essentially optically transparent” is understood to mean that the sum of transmitted and reflected light and, if applicable, light in-coupled into a carrier or layer and guided therein amounts to a minimum of 95% of the delivered light at the point of incidence of the delivered light.

The essentially optically transparent carrier preferably comprises a material from the group comprising moldable, sprayable or millable plastics, metals, metal oxides, silicates, such as glass, quartz or ceramics.

It is also preferred if the immobilization surface according to the invention is itself essentially optically transparent.

Preferably, the immobilization surface (as a PLL-g-PEG layer) has a thickness of less than 200 nm, preferably of less than 20 nm.

It is characteristic for specific embodiments that the surface for immobilization is deposited on a solid carrier, in the surface of which are structured recesses for generation of sample compartments. Thereby, these recesses in the surface of the carrier preferably have a depth of 20 μm to 500 μm, especially preferable of 50 μm to 300 μm.

Embodiments of an immobilization surface according to the invention are preferred, which are characterized in that the essentially optically transparent carrier comprises a continuous optical waveguide or an optical waveguide divided into individual waveguiding areas. It is especially preferred if the optical waveguide is an optical film waveguide with a first essentially optically transparent layer (a) facing the immobilization surface on a second essentially optically transparent layer (b) with a refractive index lower than that of layer (a). It is also preferred if said optical film waveguide is essentially planar.

It is characteristic of such an embodiment of an immobilization surface on an optical film waveguide as a carrier that, for the in-coupling of excitation light into the optically transparent layer (a), this layer is in optical contact with one or more optical in-coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

Thereby it is preferred that the excitation light is in-coupled into the optically transparent layer (a) using one or more grating structures (c) which are featured in the optically transparent layer (a). It is also preferred that out-coupling of light guided in the optically transparent layer (a) is performed using one or more grating structures (c′) which are featured in the optically transparent layer (a) and have the same or different period and grating depth as grating structures (c).

Further planar optical film waveguides and modifications thereof which are suitable as carriers of an immobilization surface according to the invention are described for example in patent applications WO 95/33197, WO 95/33198, WO 96/35940, WO 98/09156, WO 99/40415, PCT/EP 00/04869 and PCT/EP 01/00605. The content of these patent applications is therefore introduced in its entirety as an integral part of this description.

Especially preferred are such embodiments of an immobilization surface according to the invention, wherein the nucleic acids immobilized thereon as recognition elements are arranged in discrete (laterally separated) measurement areas. Up to 1,000,000 measurement areas may be provided in a 2-dimensional arrangement, and a single measurement area may cover an area of 10⁻⁴ mm²-10 mm². It is preferred that the measurement areas are arranged in a density of more than 10, preferably more than 100, especially preferably more than 1000 measurement areas per square centimeter.

The discrete (laterally separated) measurement areas may be generated on said immobilization surface by the laterally selective application of nucleic acids as recognition elements, preferably using one or more methods from the group of methods comprising ink-jet spotting, mechanical spotting by means of pin, pen or capillary, micro-contact printing, fluidic contact of the measurement areas with the biological or biochemical or synthetic recognition elements through their application in parallel or intersecting microchannels, upon exposure to pressure differences or to electric or electromagnetic potentials, and photochemical or photolithographic immobilization methods.

A further subject of the invention is a method for the simultaneous or sequential, qualitative and/or quantitative detection of one or more second nucleic acids in one or more samples, wherein said samples and if necessary further reagents are brought into contact with an immobilization surface according to any of the embodiments described hereinbefore, on which surface one or several first nucleic acids are immobilized as recognition elements for the specific binding/hybridization with said second nucleic acids, and changes in optical or electronic signals resulting from the binding/hybridization with these second nucleic acids or of further tracer substances used for analyte detection are measured.

It is preferred that the one or more samples are pre-incubated with a mixture of the various tracer reagents for determining the second nucleic acids to be detected in said samples, and these mixtures are then brought into contact with the first nucleic acids immobilized on an immobilization surface according to the invention in a single addition step. Thereby it is preferred that the detection of the one or more second nucleic acids is based on the determination of the change in one or more luminescences.

There are different optical excitation configurations which can be applied for luminescence excitation. One possibility consists in delivering the excitation light from one or more light sources, for excitation of one or more luminescences, in an epi-illumination configuration.

Characteristic for another possible configuration is that the excitation light from one or more light sources for the excitation of one or more luminescences is delivered in a transillumination configuration.

Such an embodiment of the method according to the invention is preferred wherein the immobilization surface is arranged on an optical waveguide which is preferably essentially planar, wherein one or more samples with second nucleic acids to be detected therein and, if necessary further tracer reagents, are brought sequentially or in a single addition step after mixture with said tracer reagents, into contact with said first nucleic acids immobilized as recognition elements on an immobilization surface according to the invention, and wherein the excitation light from one or more light sources is in-coupled into the optical waveguide using one or more optical coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

Characteristic for another preferred embodiment of the method according to the invention is that the detection of one or more second nucleic acids is performed on a grating structure (c) or (c′) formed in the layer (a) of an optical film waveguide, based on changes in the resonance conditions for the in-coupling of excitation light into layer (a) of a carrier formed as film waveguide or for out-coupling of light guided in layer (a), these changes resulting from binding/hybridization of said second nucleic acids or further tracer reagents to the first nucleic acids immobilized as recognition elements in the region of said grating structure on an immobilization surface according to the invention.

It is especially preferred if said optical waveguide is provided as an optical film waveguide with a first optically transparent layer (a) on a second optically transparent layer (b) with lower refractive index than layer (a), wherein excitation light is further in-coupled into the optically transparent layer (a) with the aid of one or more grating structures, which are featured in the optically transparent layer (a), and delivered as a guided wave to measurement areas (d) located thereon, and wherein the luminescence of molecules capable of luminescence, generated in the evanescent field of said guided wave, is further determined using one or more detectors, and the concentration of one or more nucleic acids to be detected is determined from the intensity of these luminescence signals.

Thereby, (1) the isotropically emitted luminescence or (2) luminescence in-coupled into the optically transparent layer (a) and out-coupled via grating structure (c) or (c′) or, simultaneously, luminescences of both (1) and (2) may be measured.

It is preferred that a luminescence dye or luminescent nanoparticle is used as luminescence label for luminescence generation, which label can be excited and emits at a wavelength between 300 nm and 1100 nm.

The luminescence label may be bound to the second nucleic acids themselves to be detected as analytes or, in a competitive assay, to nucleic acids with the same sequence as said second nucleic acids to be detected and added to the sample as competitors at a known concentration, or, in a multistep assay, to one of the binding partners of the first nucleic acids immobilized as recognition elements, or to said immobilized first nucleic acids themselves. As a multi-step assay is is here understood that not only a single second nucleic acid (as the analyte) with a sequence at least partially complementary to the sequence of the corresponding first nucleic acid is bound or hybridized, respectively, to the immobilized first nucleic acids, but that, for example, further nucleic acids are bound to these second nucleic acids.

It is characteristic for special embodiments of the method according to the invention that a second luminescence label or further luminescence labels are used with excitation wavelengths either the same as or different from that of the first luminescence label and the same or different emission wavelength. Such embodiments may be designed in such a way, upon the corresponding selection of the spectral properties of the applied luminescence labels, that the second or further luminescence labels can be excited at the same wavelength as the first luminescence label, but emit at different wavelengths.

For certain applications, for example for measurements independent of each other, applying different excitation and detection labels, it is advantageous if the excitation spectra and emission spectra of the luminescence dyes used overlap only little or not at all.

For another special embodiment of the method it is characteristic that charge or optical energy transfer from a first luminescence label serving as donor to a second luminescence label serving as acceptor is used for the purpose of detecting the second nucleic acids as analytes.

Characteristic for another special embodiment of the method according to the invention is that changes in the effective refractive index on the measurement areas are determined in addition to the determination of one or more luminescences.

It is advantageous if the one or more luminescences and/or determinations of light signals at the excitation wavelength are carried out using a polarization-selective procedure. It is especially preferred if the one or more luminescences are measured at a polarization different from that of the excitation light.

The method according to the invention is characterized in that the samples to be analyzed may be aqueous solutions, especially buffer solutions, or naturally occurring body fluids such as blood, serum, plasma, urine or tissue fluids. A sample to be analyzed may also be an optically turbid fluid, surface water, a soil or plant extract, a biological or synthetic process broth. The samples to be analyzed may also be prepared from biological tissue parts or cells.

A further subject of the invention is the use of an immobilization surface according to the invention and/or a method according to the invention for quantitative or qualitative analyses in screening methods in pharmaceutical research, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein-DNA interactions, for the determination of control mechanisms for mRNA expression and for protein (bio)synthesis, for the generation of toxicity studies and the determination of expression profiles, especially for the determination of biological and chemical marker compounds, such as mRNA, pathogens or bacteria in pharmaceutical product research and development, human and veterinary diagnostics, agrochemical product research and development, for symptomatic and pre-symptomatic plant diagnostics, for patient stratification in pharmaceutical product development and for therapeutic drug selection, for the determination of pathogens, nocuous agents and germs, especially of salmonella, prions, viruses and bacteria, especially in food and environmental analytics.

The invention will be further explained by the following example.

### EXAMPLE

1. Chemicals

1.1. Buffer Solutions

The following buffer solutions were used:

Buffer 1:

4×SSC (600 mM NaCl 160 mM sodium citrate, pH 7.5)
   Buffer 2:
4×SSC (600 mM NaCl/60 mM sodium citrate, pH 7.5) comprising 50% formamide
   Washing Buffer 1:
1×SSC (150 mM NaCl/15 mM sodium citrate, pH 7.5) comprising 0.1% SDS
   Washing Buffer 2:
0.1×SSC (15 mM NaCl/1.5 mM sodium citrate, pH 7.5) comprising 0.1% SDS
   Washing Buffer 3:
0.1×SSC (15 mM NaCl/1.5 mM sodium citrate, pH 7.5)
   1.2. First Nucleic Acids to be Immobilized

A mouse brain “longmer set”, derived from 96 genes (Lion Bioscience, Heidelberg, Germany), representing low to medium expressed genes from brain tissue of a mouse, were used, being provided as oligonucleotides of a length of 70 nucleotides each (“longmers”), the sequence of which was selected by Operon (Alamada, Calif., USA) from the sequences of said genes and was also produced by Operon.

1.3. Second Nucleic Acids to be Detected as Analyte

Starting from mouse brain, the total RNA was isolated using the kit RNeasy (QIAGEN, Hilden, Germany). In a further step, mRNA was isolated from this isolate of total RNA using the kit Oligotex (QIAGEN, Hilden, Germany). Then the mRNA isolate was utilized as a template for reverse transcription (by means of Reverse Transcriptase Omniscript, QIAGEN, Hilden, Germany). Using a poly (dT) primer, all mRNA molecules with a poly (dA) tail were transcribed to cDNA. Nucleotides fluorescently labeled with Cy5 (Amersham, Arlington Heights, USA) were applied for this transcription step, resulting in fluorescently labeled cDNA.

Dependent on the yield of mRNA isolation and the efficiency of reverse transcription, the labeled cDNA does represent the whole variety of mRNA expressed in the mouse brain used.

1.4. Production of poly(L-lysine)-g-poly(ethyleneglycol) (PLL-g-PEG)

Materials

Poly(L-lysine) hydrobromide (molecular weight about 20 kDa) was obtained from Sigma-Aldrich (Buchs, switzerland). The N-hydroxy succinimidyl ester of methoxy poly(ethyleneglycol) propionic acid (MeO-PEG-SPA, molecular weight 2 kDA) was obtained from Shearwater Polymers Inc. (Huntsville, USA). 4-(2-hydroxyethyl) piperazine-1-ethane sulfonic acid (HEPES) and further chemicals for the preparation of buffers were purchased from Fluka (Buchs, Switzerland).

All aqeuous solutions were produced using ultra-pure water (18 MOhm cm) from an “Easy Pure reverse Osmosis System” (Barnstead Thermolyne, Dubuque, USA).

Synthesis of PLL-G-PEG

The synthesis of PLL-g-PEG has been described by Sawhney and Hubbell (A. S. Sawhney, J. A. Hubbell, Biomaterials 13 (1992) 863-870). The studies which serve as a basis for the present application used procedures based on a method developed by Elbert and Hubbell (D. L. Elbert, J. A. Hubbell, J. Biomed. Mater. Res. 42 (1998) 55-65).

N-Hydroxysuccinimidyl ester of poly(ethyleneglycol) (“PEG”) is reacted with poly(L-lysine) (“PLL”) under stoichiometric conditions to manufacture the desired product. The details on this synthesis are described hereinafter.

The nomenclature used hereinafter to describe the various PLL-g-PEG derivatives includes the molecular weights of the polymer sub-chains of the copolymers and the grafting ratio. Accordingly, “PLL(20)-g[3.5]-PEG(2)” describes a polymer composed of a main chain of poly(L-lysine) with a molecular weight of 20 kDa and side chains comprising poly(ethyleneglycol) with a molecular weight of 2 kDa. The grafting ratio of 3.5 means that, on average, PEG chains in each case are bound to two of seven lysine groups (lysine units). Since all the polymers mentioned in this example were manufactured from identical precursor products, the abbreviation “PLL-g[3,7]-PEG” is also to be used as an alternative to “PLL(20)-g[3,7]-PEG(2)”.

Poly(L-lysine)hydrobromide (“PLL-HBr”) is dissolved in 25 ml sodium tetraborate buffer (“STBB”, 50 mM, pH 8.5) per gram PLL-HBr. The solution is stirred, then filtered (0.22 μm Durapore membrane, sterile Millex GV, Sigma-Aldrich, Buchs, Switzerland) and filled into a sterile culture tube. While the solution is constantly stirred, a suitable quantity of MeO-PEG-SPA powder is then added according to stoichiometric conditions. After a further six hours of stirring, the solution is transferred at room temperature to a dialysis tube (Spectr/Por dialysis tubes, molecular weight cut-off 6-8 kDa, Sochochim, Lausanne, Switzerland). The dialysis is carried out for 24 hours in a liter of phosphate-buffered saline (“PBS”, 10 mM, pH 7.0), followed by a another 24 hours of further dialysis in a liter of deionized water. The product is then lyophilized for 48 hours.

The control of the grafting ratio is performed using 1H-NMR. 6 different polymers with grafting ratios of 3.7, 7.4, 8.4, 9.0, 11.8, and 13.0 are produced as described hereinbefore.

2. Carrier

As a carrier of an immobilization surface according to the invention a planar optical film waveguide is used with the external dimensions of 57 mm in width (parallel to the grating lines of a grating structure (c) modulated in layer (a) of the film waveguide)×14 mm in length (perpendicular to the grating structures)×0.7 mm in height. 6 microflow cells can be created in the pattern of part of a column of a standard microtiter plate (9 mm spacing) by combination with a polycarbonate plate featuring open cavities in the direction of the sensor platform with the internal dimensions of 5 mm wide×7 mm long×0.15 mm high, either directly on the surface of layer (a) or after deposition of further layers, especially of an immobilizattion surface according to the invention, on layer (a). The polycarbonate plate may be adhered to the carrier in such a way that the cavities are then tightly sealed against each other. This polycarbonate plate is constructed such that it can be joined together with a substrate (“meta-carrier”) with the basic dimensions of standard microtiter plates in such a way that the pitch (arrangement of rows or columns) of the inlets of the flow cells matches the pitch of the wells of a standard microtiter plate.

The substrate material (optically transparent layer (b) of the planar optical film waveguide as a carrier) comprises AF 45 glass (refractive index n=1.52 at 633 nm). The substrate features a pair of in-coupling and out-coupling gratings with grating lines (318 nm period) running parallel with the width of the sensor platform at a grating depth of 12±3 nm, wherein the grating lines are drawn over the whole width of the film waveguide. The distance between the two consecutive gratings is 9 mm, and the length of the individual grating structures (parallel with the length of the sensor platform) is 0.5 mm. The distance between the in-coupling and out-coupling grating of a grating pair is selected such that the excitation light in each case can be in-coupled within the region of the sample compartments, after combination of the sensor platform with the aforementioned polycarbonate plate, whereas the out-coupling takes place outside the region of the sample compartment. The wave-guiding, optically transparent layer (a) comprising Ta₂O₅ on the optically transparent layer (b) has a refractive index of 2.15 at 633 nm (layer thickness 150 nm).

To prepare for immobilization of the biochemical or biological or synthetic recognition elements, the optical film waveguide as a carrier is cleaned using organic and inorganic reagents (e.g. propanol and sulfuric acid, with intermediate washing steps with water) in an ultrasonication device.

3. Generation of the Immobilization Surface

A solution of PLL-g-PEG in PBS buffer (1 mg/ml) is produced and filtered through 0.22 μm Durapore menbranes. Instead of PBS buffer, for example, also HEPES buffer can be used. 570 μl of the PLL-g-PEG solution are pipetted into a special incubation chamber for the coating of the carrier as described in section 2. of this example. Then the carriers are inserted into the incubation chamber in such a way that the surface to be coated, i.e. the surface of the layer (a) on the example of a planar optical film waveguide as a carrier to be coated, gets into contact with the polymer solution. After a two-hours incubation at room temperature, the coated carriers are rinsed with ultra-pure water and blown dry with nitrogen.

4. Immobilization of the First Nucleic Acid/Generation of Discrete Measurement Areas

The 96 oligonucleotides with a length of 70 nucleotides each, at a concentration of 40 μM in 10 mM carbonate buffer (pH 9.2, with an addition of 5% DMSO), as described in section 1.2, are deposited as biological recognition elements on the immobilization surface generated as described above using a commercial spotter (GMS 417 Arrayer, Affymetrix, Santa Clara. CA, USA) and incubated over night. The distance between the measurement areas (spots) thus generated is 340 μm. In one array always two spots with identical base sequence are generated, a single array thus comprising 192 spots. Up to 6 similar arrays are generated on a film waveguide as a carrier, according to section 2.

Arrays of immobilized first nucleic acids are generated in a similar manner on the six carriers with immobilization surfaces of different grafting ratio.

The polycarbonate plate described above is joined with the carrier coated with the immobilization surface, comprising the first nucleic acids deposited on the immobilization surface, in such a way that the individual sample compartments are fluidically sealed against one another and the generated “longmer” arrays, together with the corresponding in-coupling grating (c), are arranged each within one of the 6 sample compartments.

5. Hybridization Assay as an Integral Part of the Method According to the Invention for the Determination of One or More Second Nucleic Acids

The carrier provided with discrete measurement areas on a deposited immobilization surface according to the invention, and provided as a planar optical film waveguide, joined with a polycarbonate plate for generation of 6 sample compartments (“chambers”) according to section 2, of this example, is inserted into a “meta-carrier”. For purposes of moistening/equilibration the two-dimensional arrangements of measurement arrays (“microarrays”) are filled with 90 μl buffer 1.

A sample of the second nucleic acids (“target probe”) for hybridization to be detected as analyte is prepared from labeled cDNA (according to section 1.3) at an amount corresponding to 25 ng mRNA. An amount of cDNA in 50 μl hybridization buffer (buffer 2), corresponding to an amount of 25 ng mRNA, is added by pipetting. For purposes of denaturation, the target probe is heated to 95° C. for 5 min and then stored on ice for 5 min. Buffer 1 is evacuated from the chambers, and the target probe is pipetted upon avoiding air bubbles.

For hybridization, the “meta carrier” is inserted into a thermocycler (MJ Research PCT-200 with an adapter plate) for 35 min at 75° C. (step of denaturation) and incubated then for 18 hours at 42° C. (hybridization step).

After termination of the hybridization, the following washing steps are performed: The chambers are evacuated by application of vacuum, then filled with 90 μl buffer 1 and then temperature-equilibrated at room temperature in the “meta carrier”.

Then the chambers are evacuated again, filled with 90 μl washing buffer 1 and incubated for 7 min at room temperature. In a similar way, evacuation and filling is repeated using once washing buffer 2 and twice washing buffer 3, Finally, the chambers are evacuated and filled with buffer 1.

The hybridization assay as described above is performed in a similar way with all 6 carriers comprising immobilization surfaces of different grafting ratios.

6. Analytical System and Measurement Method for the Detection of One or More Analytes

The excitation light from a laser diode with emission at 635 nm is expanded to a ray bundle of slit-type cross section (perpendicular to the optical axis) using a lens system comprising a cylindrical lens and a diaphragm, the size of the ray bundle in the cross-section of light irradiated onto the planar optical film waveguide, in parallel to the grating lines, corresponding almost exactly to the section of the in-coupling grating located within a sample compartment.

The angle between the incoming, parallel excitation light bundle and the plane of the planar optical film waveguide is adjusted to the resonance angle for maximum in-coupling into the waveguiding layer (a) (−110), as well as the corresponding optimum position of the excitation light to be in-coupled on the in-coupling grating (first grating). This optimization is performed in an automated manner, wherein the light out-coupled by the second grating located outside of the sample compartment is directed to a photodiode, the signal of which is amplified in an adequate way and wherein the photodiode signal is optimized to a maximum value, based on the principle of a “feedback loop”, upon further adjustments of the carrier with respect to the coupling angle and the lateral position.

Light emanating from the microarray, from the region of the measurement surface within a sample compartment on the carrier provided as a planar optical film waveguide (image area about 6 mm×8 m), is collected by a tandem objective and focussed onto a CCD camera comprising a CCD chip (active area about 5 mm×7 mm with 766 pixels, pixel size: 9 μm). Dependent on the imaging system, this configuration enables a lateral resolution of about 10 μm to 20 μm.

An interference filter (670 DF 40, Omega Optical, Brattleborough, Vt., USA) is positioned between the two halves of the tandem objective, in an essentially parallel (i.e. less than 10° divergent or convergent) part of the emission ray path, for collection of the light emanating from the array at the fluorescence wavelength of the applied fluorescence label (Cy 5).

After accomplished hybridization of the immobilized first nucleic acids with the second, fluorescently labeled nucleic acids supplied as the sample, in each case the emission light from all measurement areas located within a sample compartment is collected as one image by a cooled CCD camera.

7. Analysis of the Measurement Data

The medium signal intensity from the measurement areas, for the binding and detection of analyte molecules due to a potentially generated fluorescence of fluorescence labels (Cy5 according to the example in hand) is determined using image analysis software.

The raw data obtained from the individual pixels of the camera form a two-dimensional matrix of the digitized measurement data, with the measured intensity as the measurement value of a pixel corresponding to the surface section of the sensor platform imaged onto said pixel. For data analysis, at the beginning a two-dimensional (coordinate) net is superimposed over the image points (pixel values) in such a way that each spot is contained in an individual, two-dimensional net element. Within this net element, an “analysis element” (area of interest, “AOI”) is assigned to each spot, with a geometry optimized for matching the spot geometry. These AOIs can have any geometric form, for example circular form. The location of the AOIs in the two-dimensional net is individually optimized as a function of the signal intensity recorded by the corresponding pixels. Dependent on the definitions set by the user, the initially defined radius of an AOI can be preserved or can be re-adjusted according to the geometry and size of a given spot. For example, the arithmetic average of the pixel values (signal intensities) can be determined as the mean gross signal intensity of every spot.

The background signals are determined from the signal intensities measured between the spots. For this purpose, for example, further circles can be defined, which are concentric with a given circular spot (and the assigned “spot AOI”), but have a larger radius. Of course, the radii of these concentric circles have to be smaller than the distance between adjacent spots. Then, for example, the region between the “spot AOI” and the first larger concentric circle can be disregarded, and the region between said first larger and a second still larger concentric circle can be defined as the AOI for the background determination (“background AOI”). It is also possible to define regions between adjacent spots, preferably located in the middle between adjacent spots, as AOIs for the determination of the background signal intensities. From these signal values the average background signal can then be determined in analogous way as described above, for example as the arithmetic average of the pixel values (signal intensities) of the chosen “background AOI”. The average net signal intensity can then be determined as the difference between the local average gross and the local average background signal intensity.

8. Results

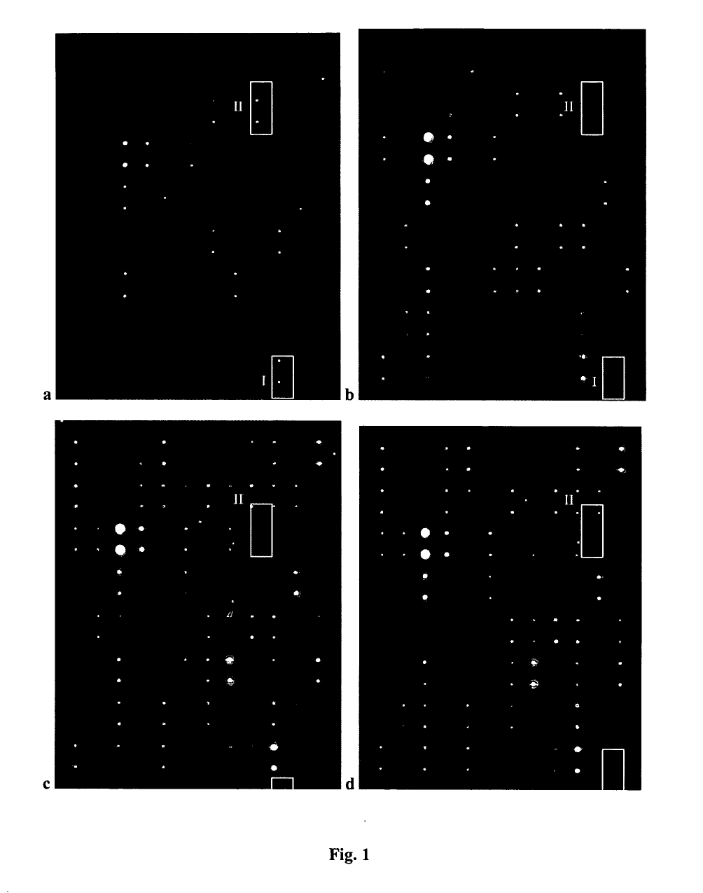

For all 6 carriers with immobilization surfaces of different grafting ratio g, the fluorescence signals from the measurement areas (“spots”) of the arrays were measured in the analytical system after termination of the hybridization assays (according to section 6. of this example). Images of the fluorescence signals determined for 4 g values, namely 3.7, 7.4, 9.0, and 11.8, are shown in FIG. 1a-1d. For the determination of the net fluorescence signals, as the difference between the gross fluorescence signals (arithmetic mean of the pixel values in the AOIs) and the background signals, according to section 7 of this example, the signals from two spot pairs (duplicates) each, as an example for the fluorescence signals after hybridization with cDNA from highly expressed genes (spot group I in FIG. 1a-d), weaker expressed genes (spot group II in FIG. 1a-d), were analyzed (marked in FIG. 1a-d). The strong effect of the grafting ratio g on the signal intensities is already evident from the comparison of images 1a-1d, which are all displayed in the same dynamic range: Many spots are clearly visible in FIG. 1d (g=1.8), visible in FIG. 1c (g=9.0), hardly visible in FIG. 1b (g=7.4), and not visible in FIG. 1a (g=3.7). This concerns, for example, the complete left row of spots.

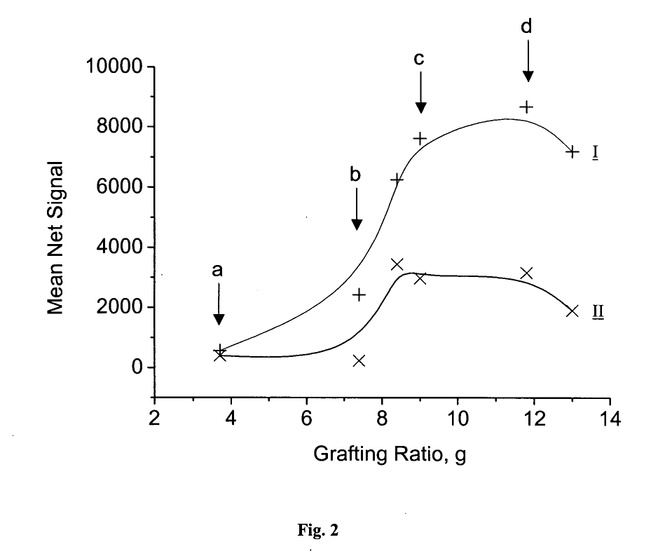

The calculated net fluorescence intensities, as average values of the signals from always two spots of similar type, are displayed in FIG. 2 as a function of the grafting ratio. For both selected spot pairs, the fluorescence signals are relatively low in the region of g=3.7 to g=7.4. Starting from g=8.4, a strong increase of the fluorescence signals is observed. With further increase of g, am extended flat region of high signal intensities (“plateau”) is reached, before the signal intensities decrease at values of g>11.8.

Based on these results it is concluded that for immobilization surfaces of the kind at hand, for achieving net signals as high as possible in hybridization assays as described herein, the grafting ratio g should have a value between 7 and 13, preferably between 8 and 12.

1. A surface for the immobilization of one or several first nucleic acids as recognition elements (“immobilization surface”), for the production of a recognition surface for the detection of one or several second nucleic acids in one or more samples which are brought into contact with the recognition surface, the first nucleic acids being applied to a layer of PLL-g-PEG (graft copolymer poly(L-lysine)-g-poly(ethyleneglycol)) as a surface for immobilization, characterized in that the grafting ratio g, in other words the ratio between the number of lysine units and the number of polyethylene glycol side chains (“PEG” side chains) has an average value between 7 and 13.

2. A surface for the immobilization of one or several first nucleic acids according to claim 1, wherein the grafting ratio g has a medium value between 8 and 12.

3. A surface for the immobilization of one or several first nucleic acids according to claim 1, wherein the molecular weight of the polyetheyleneglycol side chains (“PEG” side chains) is between 500 Da and 7000 Da.

4. A surface for the immobilization of one or several first nucleic acids according to claim 1, wherein the molecular weight of the polyetheyleneglycol side chains (“PEG” side chains) is between 1500 Da and 5000 Da.

5. A surface for the immobilization of one or several first nucleic acids according to claim 1, wherein said surface is deposited on a solid carrier.

6. A surface for the immobilization of one or several first nucleic acids according to claim 5, wherein said solid carrier is an essentially optically transparent carrier.

7. A surface for the immobilization of one or several first nucleic acids according to claim 6, wherein the essentially optically transparent carrier comprises a material from the group comprising moldable, sprayable or millable plastics, metals, metal oxides, silicates, such as glass, quartz or ceramics.

8. A surface for the immobilization of one or several first nucleic acids according to claim 1, wherein said surface is essentially optically transparent.

9. A surface for the immobilization of one or several first nucleic acids according to claim 1, wherein said surface (as a PLL-g-PEG layer) has a thickness of less than 200 nm, preferably of less than 20 nm.

10. An immobilization surface according to claim 6, wherein said surface for immobilization is deposited on a solid carrier, in the surface of which are structured recesses for generation of sample compartments.

11. An immobilization surface according to claim 10, wherein said recesses in the surface of the carrier have a depth of 20 μm to 500 μm, especially preferably 50 μm to 300 μm.

12. An immobilization surface according to claim 6, wherein the essentially optically transparent carrier comprises a continuous optical waveguide or an optical waveguide divided into individual waveguiding areas.

13. An immobilization surface according to claim 12, wherein the optical waveguide is an optical film waveguide with a first essentially optically transparent layer (a) facing the immobilization surface on a second essentially optically transparent layer (b) with a refractive index lower than that of layer (a).

14. An immobilization surface according to claim 13, wherein said optical film waveguide is essentially planar.

15. An immobilization surface according to claim 13, wherein, for the in-coupling of excitation light into the optically transparent layer (a), this layer is in optical contact with one or more optical in-coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

16. An immobilization surface according to claim 15, wherein the excitation light is in-coupled into the optically transparent layer (a) using one or more grating structures (c) which are featured in the optically transparent layer (a).

17. An immobilization surface according to claim 15, wherein light guided in the optically transparent layer (a) is out-coupled using one or more grating structures (c′) which are featured in the optically transparent layer (a) and have the same or different period and grating depth as grating structures (c).

18. An immobilization surface according to claim 1, wherein the nucleic acids immobilized thereon as recognition elements are arranged in discrete (laterally separated) measurement areas.

19. An immobilization surface according to claim 18, wherein up to 1,000,000 measurement areas are provided in a 2-dimensional arrangement and a single measurement area covers an area of 10⁻⁴ mm²-10 mm².

20. An immobilization surface according to claim 18, wherein the measurement areas are arranged in a density of more than 10, preferably more than 100, especially preferably more than 1000 measurement areas per square centimeter.

21. An immobilization surface according to claim 18, wherein discrete (laterally separated) measurement areas are generated on said immobilization surface by the laterally selective application of nucleic acids as recognition elements, preferably using one or more methods from the group of methods comprising ink-jet spotting, mechanical spotting by means of pin, pen or capillary, micro-contact printing, fluidic contact of the measurement areas with the biological or biochemical or synthetic recognition elements through their application in parallel or intersecting microchannels, upon exposure to pressure differences or to electric or electromagnetic potentials, and photochemical or photolithographic immobilization methods.

22. A method for the simultaneous or sequential, qualitative and/or quantitative detection of one or more second nucleic acids in one or more samples, wherein said samples and if necessary further reagents are brought into contact with an immobilization surface according to claim 1, on which surface one or several first nucleic acids are immobilized as recognition elements for the specific binding/hybridization with said second nucleic acids, and changes in optical or electronic signals resulting from the binding/hybridization with these second nucleic acids or of further tracer substances used for analyte detection are measured.

23. A method according to claim 22, wherein the one or more samples are pre-incubated with a mixture of the various tracer reagents for determining the second nucleic acids to be detected in said samples, and these mixtures are then brought into contact with the first nucleic acids immobilized on said immobilization surface in a single addition step.

24. A method according to claim 22, wherein the detection of the one or more second nucleic acids is based on the determination of the change in one or more luminescences.

25. A method according to claim 22, wherein the excitation light from one or more light sources for the excitation of one or more luminescences is delivered in an epi-illumination configuration.

26. A method according to claim 22, wherein the excitation light from one or more light sources for the excitation of one or more luminescences is delivered in a transillumination configuration.

27. A method according to one of claims 22-24 claim 22, wherein the immobilization surface is arranged on an optical waveguide which is preferably essentially planar, wherein one or more samples with second nucleic acids to be detected therein and, if necessary further tracer reagents, are brought sequentially or in a single addition step after mixture with said tracer reagents, into contact with said first nucleic acids immobilized as recognition elements on said immobilization surface, and wherein the excitation light from one or more light sources is in-coupled into the optical waveguide using one or more optical coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

28. A method according to claim 27, wherein the detection of one or more second nucleic acids is performed on a grating structure (c) or (c′) formed in the layer (a) of an optical film waveguide, based on changes in the resonance conditions for the in-coupling of excitation light into layer (a) of a carrier formed as film waveguide or for out-coupling of light guided in layer (a), these changes resulting from binding/hybridization of said second nucleic acids or further tracer reagents to the first nucleic acids immobilized as recognition elements in the region of said grating structure on said immobilization surface.

29. A method according to claim 27, wherein said optical waveguide is designed as an optical film waveguide with a first optically transparent layer (a) on a second optically transparent layer (b) with lower refractive index than layer (a), wherein excitation light is further in-coupled into the optically transparent layer (a) with the aid of one or more grating structures, which are featured in the optically transparent layer (a), and delivered as a guided wave to measurement areas (d) located, and wherein the luminescence of molecules capable of luminescence, generated in the evanescent field of said guided wave, is further determined using one or more detectors, and the concentration of one or more nucleic acids to be detected is determined from the intensity of these luminescence signals.

30. A method according to claim 29, wherein (1) the isotropically emitted luminescence or (2) luminescence in-coupled into the optically transparent layer (a) and out-coupled via grating structure (c) or (c′) or luminescences of both (1) and (2) are measured simultaneously.

31. A method according to claim 29, wherein, for the generation of luminescence, a luminescence dye or luminescent nanoparticle is used as a luminescence label, which can be excited and emits at a wavelength between 300 nm and 1100 nm.

32. A method according to claim 31, wherein the luminescence label is bound to the second nucleic acids themselves to be detected as analytes or, in a competitive assay, to nucleic acids with the same sequence as said second nucleic acids to be detected and added to the sample as competitors at a known concentration, or, in a multistep assay, to one of the binding partners of the first nucleic acids immobilized as recognition elements, or to said immobilized first nucleic acids.

33. A method according to claim 31, wherein a second luminescence label or further luminescence labels are used with excitation wavelengths either the same as or different from that of the first luminescence label and the same or different emission wavelength.

34. A method according to claim 33, wherein the second or further luminescence labels can be excited at the same wavelength as the first luminescence label, but emit at different wavelengths.

35. A method according to claim 33, wherein the excitation spectra and emission spectra of the luminescence dyes used overlap only little or not at all.

36. A method according to claim 33, wherein charge or optical energy transfer from a first luminescence label serving as donor to a second luminescence label serving as acceptor is used for the purpose of detecting the second nucleic acids as analytes.

37. A method according to claim 29, wherein changes in the effective refractive index on the measurement areas are determined in addition to the determination of one or more luminescences.

38. A method according to claim 29, wherein the one or more luminescences and/or determinations of light signals at the excitation wavelength are carried out using a polarization-selective procedure.

39. A method according to claim 29, wherein the one or more luminescences are measured at a polarization different from that of the excitation light.

40. A method according to claim 22, wherein the samples to be analyzed are aqueous solutions, especially buffer solutions, or naturally occurring body fluids such as blood, serum, plasma, urine or tissue fluids.

41. A method according to claim 22, wherein the sample to be analyzed is an optically turbid fluid, surface water, a soil or plant extract, a biological or synthetic process broth.

42. A method according to claim 22, wherein the samples to be analyzed are prepared from biological tissue parts or cells.

43. Use of an immobilization surface according to claim 1 for quantitative or qualitative analyses in screening methods in pharmaceutical research, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein-DNA interactions, for the determination of control mechanisms for mRNA expression and for protein (bio)synthesis, for the generation of toxicity studies and the determination of expression profiles, especially for the determination of biological and chemical marker compounds, such as mRNA, pathogens or bacteria in pharmaceutical product research and development, human and veterinary diagnostics, agrochemical product research and development, for symptomatic and pre-symptomatic plant diagnostics, for patient stratification in pharmaceutical product development and for therapeutic drug selection, for the determination of pathogens, nocuous agents and germs, especially of salmonella, prions, viruses and bacteria, especially in food and environmental analytics.

44. An immobilization surface according to claim 8, wherein said surface for immobilization is deposited on a solid carrier, in the surface of which are structured recesses for generation of sample compartments.

45. An immobilization surface according to claim 9, wherein said surface for immobilization is deposited on a solid carrier, in the surface of which are structured recesses for generation of sample compartments.

46. An immobilization surface according to claim 8, wherein the essentially optically transparent carrier comprises a continuous optical waveguide or an optical waveguide divided into individual waveguiding areas.

47. An immobilization surface according to claim 46, wherein the optical waveguide is an optical film waveguide with a first essentially optically transparent layer (a) facing the immobilization surface on a second essentially optically transparent layer (b) with a refractive index lower than that of layer (a).

48. An immobilization surface according to claim 9, wherein the essentially optically transparent carrier comprises a continuous optical waveguide or an optical waveguide divided into individual waveguiding areas.

49. An immobilization surface according to claim 48, wherein the optical waveguide is an optical film waveguide with a first essentially optically transparent layer (a) facing the immobilization surface on a second essentially optically transparent layer (b) with a refractive index lower than that of layer (a).

50. A method for the simultaneous or sequential, qualitative and/or quantitative detection of one or more second nucleic acids in one or more samples, wherein said samples and if necessary further reagents are brought into contact with an immobilization surface according to claim 6, on which surface one or several first nucleic acids are immobilized as recognition elements for the specific binding/hybridization with said second nucleic acids, and changes in optical or electronic signals resulting from the binding/hybridization with these second nucleic acids or of further tracer substances used for analyte detection are measured.

51. A method according to claim 50, wherein the immobilization surface is arranged on an optical waveguide which is preferably essentially planar, wherein one or more samples with second nucleic acids to be detected therein and, if necessary further tracer reagents, are brought sequentially or in a single addition step after mixture with said tracer reagents, into contact with said first nucleic acids immobilized as recognition elements on said immobilization surface, and wherein the excitation light from one or more light sources is in-coupled into the optical waveguide using one or more optical coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

52. A method for the simultaneous or sequential, qualitative and/or quantitative detection of one or more second nucleic acids in one or more samples, wherein said samples and if necessary further reagents are brought into contact with an immobilization surface according to claim 8, on which surface one or several first nucleic acids are immobilized as recognition elements for the specific binding/hybridization with said second nucleic acids, and changes in optical or electronic signals resulting from the binding/hybridization with these second nucleic acids or of further tracer substances used for analyte detection are measured.

53. A method according to claim 52, wherein the immobilization surface is arranged on an optical waveguide which is preferably essentially planar, wherein one or more samples with second nucleic acids to be detected therein and, if necessary further tracer reagents, are brought sequentially or in a single addition step after mixture with said tracer reagents, into contact with said first nucleic acids immobilized as recognition elements on said immobilization surface, and wherein the excitation light from one or more light sources is in-coupled into the optical waveguide using one or more optical coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

54. A method for the simultaneous or sequential, qualitative and/or quantitative detection of one or more second nucleic acids in one or more samples, wherein said samples and if necessary further reagents are brought into contact with an immobilization surface according to claim 9, on which surface one or several first nucleic acids are immobilized as recognition elements for the specific binding/hybridization with said second nucleic acids, and changes in optical or electronic signals resulting from the binding/hybridization with these second nucleic acids or of further tracer substances used for analyte detection are measured.

55. A method according to claim 54, wherein the immobilization surface is arranged on an optical waveguide which is preferably essentially planar, wherein one or more samples with second nucleic acids to be detected therein and, if necessary further tracer reagents, are brought sequentially or in a single addition step after mixture with said tracer reagents, into contact with said first nucleic acids immobilized as recognition elements on said immobilization surface, and wherein the excitation light from one or more light sources is in-coupled into the optical waveguide using one or more optical coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

56. Use of a method according to claim 22 for quantitative or qualitative analyses in screening methods in pharmaceutical research, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein-DNA interactions, for the determination of control mechanisms for mRNA expression and for protein (bio)synthesis, for the generation of toxicity studies and the determination of expression profiles, especially for the determination of biological and chemical marker compounds, such as mRNA, pathogens or bacteria in pharmaceutical product research and development, human and veterinary diagnostics, agrochemical product research and development, for symptomatic and pre-symptomatic plant diagnostics, for patient stratification in pharmaceutical product development and for therapeutic drug selection, for the determination of pathogens, nocuous agents and germs, especially of salmonella, prions, viruses and bacteria, especially in food and environmental analytics.
